(19) United States
(12) Patent Application Publication
(10) Pub. No.: US 2009/0198160 A1
(43) Pub. Date: Aug. 6, 2009

(54) System and Method of Providing Aeration, Cooling, Heating and Treatment to Body Region Covered by an Orthopedic Cast

(75) Inventor: Steven M. Coyne, Newport Beach, CA (US)

Correspondence Address: FOUNTAIN LAW GROUP, INC., 18201 VON KARMAN AVENUE, SUITE 960, IRVINE, CA, 92612, US

(73) Assignee: THE SM COYNE COMPANY, Newport Beach, CA (US)

(21) Appl. No.: 12/024,933

(22) Filed: Feb. 1, 2008

(51) Int. Cl. A61F 5/00

(52) U.S. Cl. 602/2; 602/7

(57) ABSTRACT

An orthopedic cast for protecting an injured body part, and including an embedded tube for routing a pressurized gaseous fluid to proximate the injured body part so that the pressurized gaseous fluid or effects thereof may be applied to the injured body part. The tube may be connected to a source of pressurized gaseous fluid to allow the pressurized gaseous fluid to be introduced into the orthopedic cast. With this orthopedic cast, various treatment may be applied to the injured body part, even though the injured body part is not directly accessible. As examples, to treat discomfort due to heat and/or perspiration, ambient or cooled pressurized air may be applied to the orthopedic cast. To cause a relaxation of muscles of the injured body part, heated pressurized air may be applied to the orthopedic cast. Medicated or therapeutic pressurized gaseous fluid may also be applied to the injured area via the orthopedic cast.

### FIELD

The embodiments of the invention generally relate to orthopedic casts, and in particular, to a system and method of providing aeration, cooling, heating and/or medication to body region covered by an orthopedic cast.

### BACKGROUND

Orthopedic casts support and protect injured bones and soft tissue during the healing of the injured area. Additionally, these casts help to reduce pain, swelling, and muscle spasm. Often, these casts are applied following surgery, and are typically custom-made and administered by a doctor and/or an assistant.

Orthopedic casts typically consists of three distinct layers. The first layer typically consists of a tube made of cotton or other soft flexible material. This layer, often referred to as the stockinette, lays closest to the patient's skin and is normally the first layer to be applied. Stockinettes are applied by carefully sliding the material over the injured area. Excess material is left at the top and bottom of the stockinette to form a cuff after the third layer is applied.

The second layer typically consists of a cotton or felt padding applied over the stockinette. The padding typically comes in rolls which are wrapped around the stockinette. Several layers of padding may be needed to form a protective cushion between the stockinette and the hard third or outer layer of the cast. The padding layer should be evenly distributed so that the padding layer is uniformly thick once completed.

Typically, the third layer is formed of plaster or fiberglass and is applied over the padding layer. Generally, plaster or fiberglass rolls are soaked in water and layered around the padding layer. Several layers of plaster or fiberglass are added until a thick outer layer is formed. At this point, the excess material at the top and bottom of the stockinette is rolled back over the outer layer to form a cuff at the top and bottom of the cast. This cuff may prevent irritation and injury to the skin from the normally rough edges of the outer layer. When the outer layer dries, it hardens into a generally non-flexible protective shell effectively immobilizing the injured area. The immobilized area allows torn ligaments to heal and bone to mend in a proper alignment and reduces the risk of re-injury.

One problem with the typical cast is that it may be uncomfortable to wear. The discomfort can be most pronounced underneath the areas covered by the cast because of the extra layers of insulation and the lack of evaporative cooling. Plaster and fiberglass combined with several layers of cotton padding and one or more layers of lining do not allow the affected area to “breathe” well. As a result, the inside of a cast may be uncomfortably warm and humid from body heat and excess perspiration. The combination of moisture and warmth may also provide an ideal setting for bacteria and fungus to thrive, possibly leading to irritation, rashes and infection.

Wearing a cast for long extended periods may be uncomfortable for other reasons. Muscles already injured may be further strained by the additional weight of a cast. Movement is also restricted, thus normal stretching may be out of the question. The application of hot and cold compresses may serve to alleviate some of these symptoms. For instance, a cold compress may reduce swelling and inflammation and help reduce localized pain. A hot compress may relax muscles and sooth soreness. Within the tight confines of a cast, however, delivery of effective heat and cold therapy or medication may present a problem.

### BRIEF DESCRIPTIONS OF THE DRAWINGS

FIG. 1 illustrates a side view of an exemplary orthopedic cast in accordance with an embodiment of the invention.

FIG. 2 illustrates a top sectional view of an exemplary orthopedic cast in accordance with another embodiment of the invention.

FIG. 3 illustrates a side view of an exemplary diffuser tube as part of an orthopedic cast in accordance with another embodiment of the invention.

FIG. 4 illustrates a side view of another tube as part of an orthopedic cast in accordance with another embodiment of the invention.

FIG. 5 illustrates a side view of another exemplary orthopedic cast in accordance with another embodiment of the invention.

FIG. 6 illustrates a view an exemplary orthopedic cast system in accordance with another embodiment of the invention.

### DETAILED DESCRIPTION OF THE EXEMPLARY EMBODIMENTS

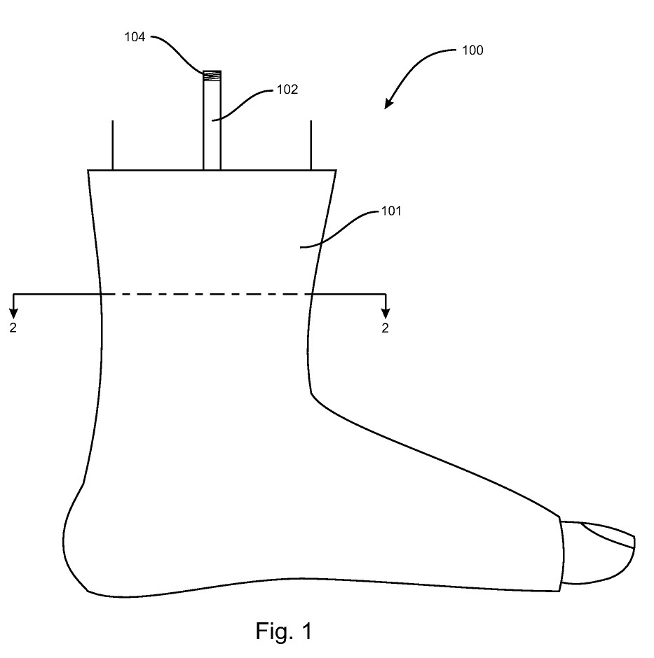

FIG. 1 illustrates a side view of an exemplary orthopedic cast 100 in accordance with an embodiment of the invention. In this example, the orthopedic cast 100 is used to protect an injured region of a human body around the lower leg, ankle, and foot. It shall be understood that the orthopedic cast 100 may be used to protect any injured area of a human body, or even any injured area of a body of an animal. In particular, the orthopedic cast 100 comprises an enclosure 101 that surrounds and protects the injured area, in this case the lower leg, ankle, and foot of a human body. The orthopedic cast 100 further comprises a tube 102 including a portion that extends outside of the enclosure 101 and a portion that extends within the enclosure 101. The tube 102 may include a port connector 104 to connect to a source of pressurized gaseous fluid, such as a pressurized air source.

The tube 102 allows gaseous fluid to be introduced into the enclosure 101 for the purpose of treating at least a portion of the injured area covered by the enclosure 101. For example, the treatment of the injured area may include applying ambient or cool pressurized air to the injured area via the tube 102 in order to cool the injured area to alleviate discomfort resulting from excess heat, sweat, skin irritation such as itching and rashes, and/or other discomforts. Alternatively, or in addition to, the treatment of the injured area may include applying heated pressurized air to the injured area via the tube 102 for administering a heat therapy to the injured area. Alternatively, or in addition to, the treatment of the injured area may include applying medicated or therapeutic gaseous fluid to the injured area via the tube 102.

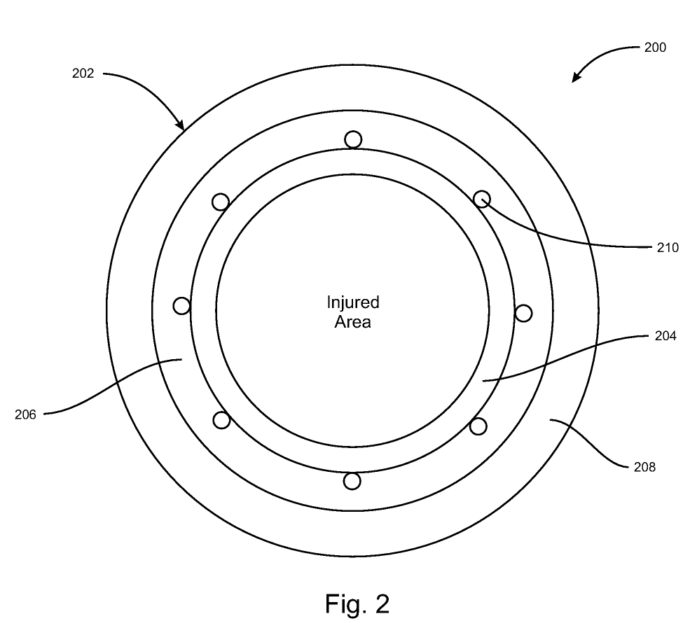

FIG. 2 illustrates a top sectional view of an exemplary orthopedic cast 200 in accordance with an embodiment of the invention. The orthopedic cast 200 comprises an enclosure 202 for enclosing and protecting an injured area of a human or animal body. The enclosure 202 may comprise a first layer 204 that completely surrounds and makes contact to the injured area. The first layer 204 may be configured to provide a comfortable feel to the injured area. For example, the first layer 204 may comprise a tube made of cotton or other soft flexible material, such as a stockinette that lays closest to the patient's skin.

The enclosure 202 further comprises a second layer 206 that completely surrounds and makes contact to the outer perimeter of the first layer 204. The second layer 206 may be configured as a cushion or padding layer to absorb energy from impact with external objects so as to protect the injured area. The second layer 206 may comprise a cotton or felt padding applied over the first layer 204, such as the stockinette. The padding layer may come in rolls which are wrapped around the stockinette. Several layers of padding may be needed to form a protective cushion between the stockinette and the hard third or outer layer of the cast. The padding layer should be evenly distributed so that the padding layer is uniformly thick once completed.

The enclosure 202 further comprises a third layer 208 that completely surrounds and makes contact to the outer perimeter of the second layer 206. The third layer 208 may be configured as a rigid or hard layer to effectively immobilize the injured area so that healing of the injured area can occur. Being rigid or hard, the third layer 208 also serves to protect the injured area from impact with external objects. The third layer 208 may be formed of plaster or fiberglass and is applied over the padding layer 206. Generally plaster or fiberglass rolls are soaked in water and layered around the padding layer. Several layers of plaster or fiberglass are added until a thick outer layer is formed. When the third layer 208 dries, it hardens into a generally non-flexible protective shell effectively immobilizing the injured area.

The orthopedic cast 200 further comprises a tube 210 for applying gaseous fluid or effects thereof to the injured area from an external source. As previously discussed, the tube 210 serves in the treatment of the injured area by routing specified gaseous fluid to injured area. As some example, the treatment may be to cool the injured area for comfort purposes and/or reduce perspiration from the injured area. In this case, an external source may apply ambient or cool pressurized air to the injured area via the tube 210. As another example, the treatment may be to heat the injured area so as to, for example, loosen the muscles of the injured area. In such a case, an external source may apply heated pressurized air to the injured area via the tube 210.

As still another example, the treatment may be to apply some medication to the injured area. For example, the medication may be for reducing skin rash or other irritation in the vicinity of the injured area. As another example, the medication may be a muscle relaxant for relaxing the muscles associated with the injured area. There could be many other types of medication applied to the injured area. In such a case, an external source may apply the medication in the form of a medicated pressurized gaseous fluid to the injured area via the tube 210.

Yet still another example, the treatment may be to apply some therapy to the injured area. The therapy may vary substantially depending on the purpose for the therapy. For example, the therapy may be to apply heat to the injured area for 10 minutes, then apply a particular medication to the injured area, and then applying a pulsing (varying pressure) ambient or cool pressurized air to the injured. In such a case, a pair of external sources, an air source and a medication source, may apply the appropriate component of the therapy to the injured area via the tube 210. The above are some examples of various applications, among many others, for which the orthopedic cast 200 may be used.

The tube 210 may be embedded within the enclosure 202 in many different ways. In the example shown in FIG. 2, the tube 210 may be situated between the first and second layers 204 and 206. The tube 210 may be further oriented to substantially run along the longitudinal axis of the orthopedic cast at various locations surrounding the injured area (in other words, to serpentine the tube 210 within the enclosure so that its main sections run along the longitudinal axis of the orthopedic casts). Other configurations are possible as discussed in more detail below.

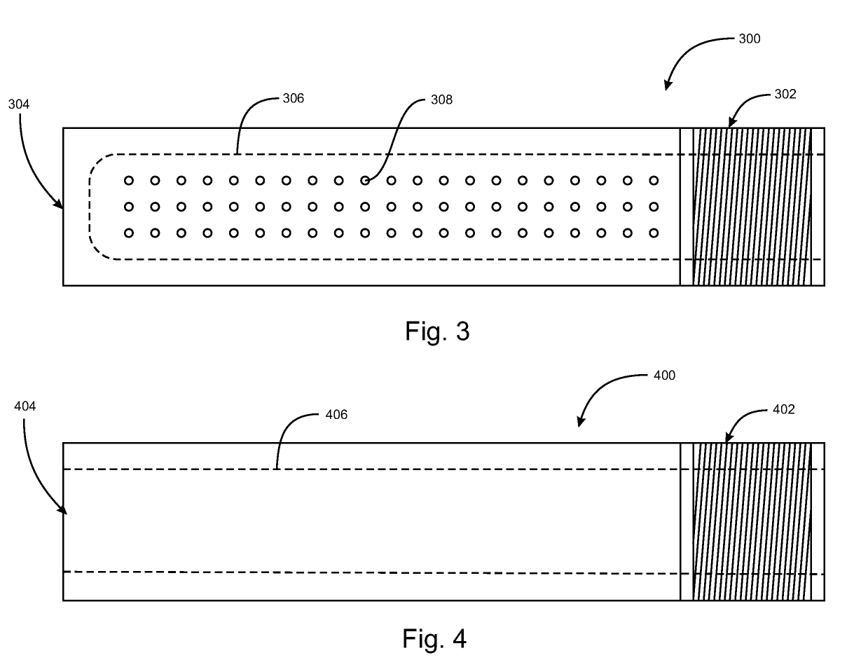

FIG. 3 illustrates a side view of an exemplary diffuser tube 300 as part of an orthopedic cast in accordance with another embodiment of the invention. The diffuser tube 300 may be made of a flexible material, such as plastic. The diffuser tube 300 comprises a connector 302 for connecting to an external source of a pressurized gaseous fluid. The diffuser tube 300 further includes an end portion 304 which is solid and does not allow gaseous fluid to flow therethrough. The diffuser tube 300 includes an internal wall 306 that defines a channel through which the gaseous fluid flows. Additionally, the diffuser tube 300 includes a plurality of orifices 308 to allow the gaseous fluid flowing through the channel to escape the tube so that it can be applied to the injured area. The orifices 308 may be situated around the entire tube, or may be positioned along a particular side of the tube. If the latter is the case, the diffuser tube 300 should be oriented inside the enclosure 202 of the orthopedic cast 200 in a manner that the orifices 308 face the injured area, so that the gaseous fluid may be directed towards the injured area.

FIG. 4 illustrates a side view of another tube 400 as part of an orthopedic cast in accordance with another embodiment of the invention. The tube 400 is more suitable for delivering effects of a gaseous fluid, such as its temperature and/or pressure, to the injured area, instead of the actual gaseous fluid itself. In particular, the tube 400 may be made of a flexible material, such as plastic. The tube 400 comprises a connector 402 for connecting to an external source of a pressurized gaseous fluid. The tube 400 further comprises an end 404 which is opened to allow the gaseous fluid to pass therethrough. Additionally, the tube 400 comprises an internal wall 406 which defines a channel through which the gaseous fluid flows. In this case, the tube 400 does not include any orifices. Thus, the treatment that can be provided to the injured area via this tube 400 is, as discussed above, effects associated with the gaseous fluid, such as temperature and pressure effects. Since the tube 400 has an outlet at its end 404, both the beginning and end portions of the tube 400 may be situated external to the enclosure of the orthopedic cast, while the remaining is situated within the enclosure to provide treatment to the injured area.

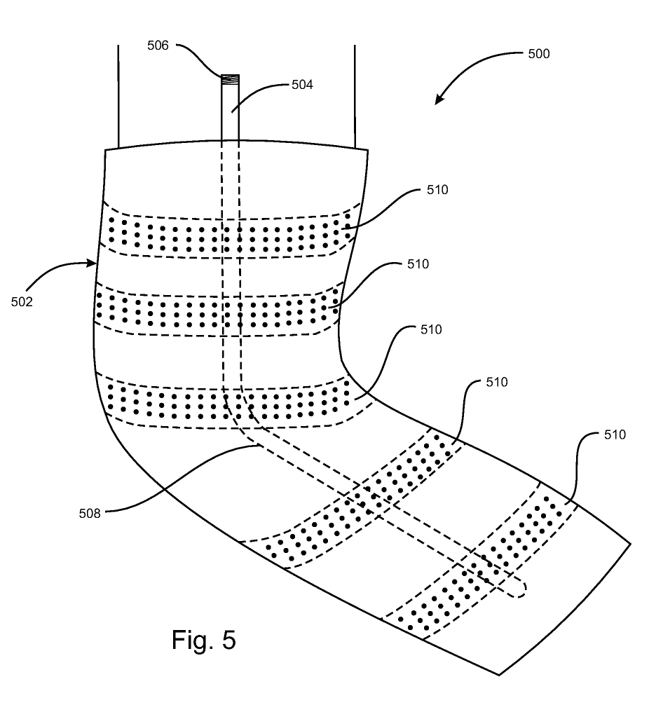

FIG. 5 illustrates a side view of another exemplary orthopedic cast 500 in accordance with another embodiment of the invention. In this embodiment, the orthopedic cast 500 covers the arm of a patient to protect and immobilize an injured elbow. The orthopedic cast comprises an enclosure 502 and a tube 504 that extends into the enclosure 502 from above. As shown, the tube 504 includes a connector 506 for connecting to a source of gaseous fluid. In this embodiment, the tube 504 includes a main portion 508 that runs longitudinally along the length of the enclosure 502. The tube 504 further includes a plurality of striped-shaped diffuser branches 510 that extend generally perpendicular from the main portion 508 at predetermined location within the enclosure 502. The main portion 508 in this embodiment may not be perforated with holes and may be capped at its lower end. The main portion 508 includes a channel fluidly coupled to each diffuser branch 510, which may be perforated with a multitude of small holes on one or both faces. The number of diffusers may depend on the length of the cast and the pressure available from the gaseous fluid external source.

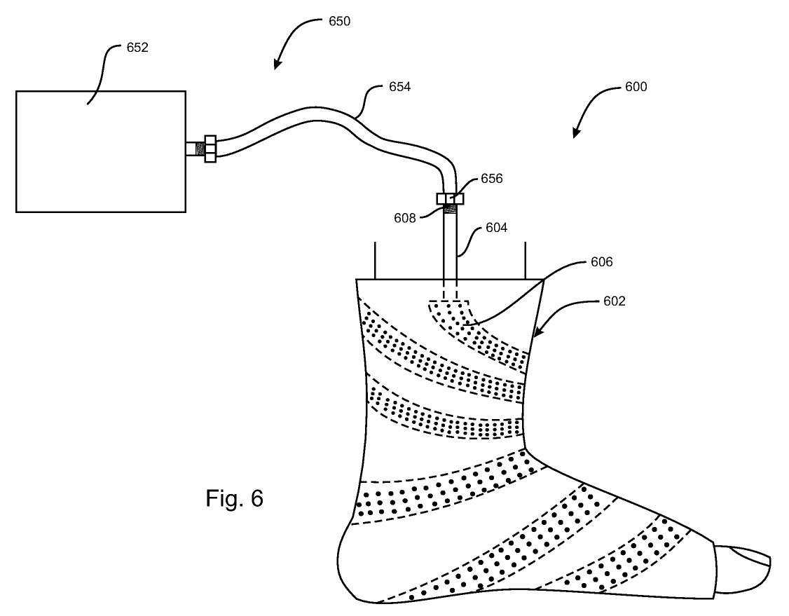

FIG. 6 illustrates a side view of an exemplary system for applying treatment in the form of a pressurized gaseous fluid to an injured area covered by an orthopedic cast in accordance with another embodiment of the invention. The system comprises a source 650 of pressurized gaseous fluid, which may comprise a pump 652 to create the pressurized gaseous fluid, a hose 654 to deliver the pressurized gaseous fluid to an orthopedic cast 600, and a hose connector 656 to connect to a corresponding connector 608 of a tube 604 of the orthopedic cast 600. The orthopedic cast 600, in turn, comprises an enclosure 602 to protect and substantially immobilize the injured area, which in this example is a lower leg, ankle and foot area. The orthopedic cast 600 further comprises a diffuser 606 fluidly coupled to the tube 604 for applying the pressurized gaseous fluid to the injured area. In this embodiment, the diffuser 606 is wrapped around the injured area in a spiral manner within the enclosure 602. The diffuser 606 may be attached to the first layer of the enclosure 602, such as the stockinette, by some form of adhesive such as surgical tape to prevent it from moving away from its intended position.

In the case that the gaseous fluid is air, the air pumped into the cast may be treated to increase its therapeutic value. For example, dehumidifying the air prior to injecting the treated air into the cast may increase the amount of moisture evaporated within the cast. The drier air should evaporate more moisture from the inside of the cast. Another example of treating the air is changing the temperature of the air before injecting the treated air into the cast. For example, the air may be cooled. Cool air may reduce swelling which could make wearing a cast more comfortable if the cast is tight. Cool air may also reduce sweating under the cast. The air may also be heated. Dry hot air increases evaporation, especially when combined with a non-absorbent lining and fiberglass outer shell. A non-absorbent lining, such as Gortex® and a fiberglass outer shell may allow moisture to pass through the layers once it evaporates. Heat treated air may therefore reduce moisture in the cast. Heat treatment also relaxes the muscles of the injured area, which may prevent cramping due to inactivity.

While the invention has been described in connection with various embodiments, it will be understood that the invention is capable of further modifications. This application is intended to cover any variations, uses or adaptation of the invention following, in general, the principles of the invention, and including such departures from the present disclosure as come within the known and customary practice within the art to which the invention pertains.

What is claimed is:

1. A system for providing pressurized gaseous fluid or effect thereof to an injured body part, comprising:
an orthopedic cast for covering and protecting the injured body part;
a source for providing the pressurized gaseous fluid; and
a tube including at least a portion situated within the orthopedic cast for routing the pressurized gaseous fluid from the pressurized gaseous fluid source to within the orthopedic cast so that the pressurized gaseous fluid or effect thereof is applied the injured body part.

2. The system of claim 1, wherein the tube comprises a plurality of orifices for dispersing the pressurized gaseous fluid within the orthopedic cast to allow the gaseous fluid to be applied to the injured body part.

3. The system of claim 2, wherein the tube comprises an occluded end adapted to prevent the gaseous fluid to flow therethrough.

4. The system of claim 1, wherein the tube comprises a connector for connecting to the pressurized gaseous fluid source.

5. The system of claim 1, wherein the tube includes a first end serving as an inlet for the pressurized gaseous fluid, and a second end serving as an outlet for the pressurized gaseous fluid.

6. The system of claim 1, wherein the orthopedic cast comprises:
a first layer that surrounds the injured body part;
a second layer that surrounds the first layer; and
a third layer that surrounds the second layer.

7. The system of claim 6, wherein the tube is situated between the first and second layers.

8. The system of claim 6, wherein the first layer comprises a stockinette, the second layer comprises a padding layer, and the third layer comprises a substantially rigid layer.

9. The system of claim 1, wherein the tube is configured to substantially spiral within the orthopedic cast.

10. The system of claim 1, wherein the tube is configured to substantially serpentine within the orthopedic cast.

11. The system of claim 1, wherein the pressurized gaseous fluid source comprises a pressurized air source adapted to provide the gaseous fluid as a pressurized air.

12. The system of claim 11, wherein the pressurized air source is adapted to provide the pressurized air with an ambient temperature, with a temperature substantially cooler than ambient temperature, or with a temperature substantially hotter than ambient temperature.

13. The system of claim 1, wherein the pressurized gaseous fluid source comprises a medicated gaseous fluid source adapted to provide the gaseous fluid as a pressurized medicated gaseous fluid.

14. A method of treating an injured body part, comprising:
forming a first layer around the injured body part;
positioning a tube adjacent to the first layer in a manner that the tube is capable of routing a pressurized gaseous fluid proximate the injured body part so that the pressurized gaseous fluid or effect thereof is applied the injured body part; and
forming a second layer around the first layer and the second layer.

15. The method of claim 14, wherein the first layer comprises a stockinette.

16. The method of claim 14, wherein the second layer comprises a padding layer.

17. The method of claim 14, wherein the tube comprises a plurality of orifices for dispersing the pressurized gaseous fluid to allow the pressurized gaseous fluid to be applied to the injured body part.

18. The method of claim 14, further comprising a third layer surrounding the second layer.

19. The method of claim 18, wherein the third layer comprises a substantially rigid layer.

20. A method of treating an injured body part, comprising:
providing an orthopedic cast having a tube for routing a pressurized gaseous fluid proximate the injured body part covered by the orthopedic cast; and
applying a pressurized gaseous fluid to the tube so that the pressurized gaseous fluid or effect thereof is applied to the injured body part.

21. The method of claim 20, wherein the pressurized gaseous fluid comprises air.

22. The method of claim 21, wherein the air is at ambient temperature, a temperature substantially cooler than ambient temperature, or a temperature substantially hotter than ambient temperature.

23. The method of claim 20, where the pressurized gaseous fluid comprises a medicated gaseous fluid.

24. The method of claim 20, further comprising varying a temperature or a pressure of the pressurized gaseous fluid in a specified manner to provide therapy to the injured body part.

25. A medical device, comprising:
an orthopedic cast for covering an injured body part; and
a tube situated within the orthopedic cast for routing a pressurized gaseous fluid from an external source of the pressurized gaseous fluid into the orthopedic cast so that the pressurized gaseous fluid or effect thereof is applied the injured body part.
